(19) United States
(12) Patent Application Publication
(10) Pub. No.: US 2025/0049388 A1
(43) Pub. Date: Feb. 13, 2025

(54) Adaptive Closed Loop Hemorrhagic Shock Resuscitation Controller

(71) Applicant: The Government of the United States, as Represented by the Secretary of the Army, Fort Detrick, MD (US)

(72) Inventors: Saul J. Vega, Fort Detrick, MD (US); Eric J. Snider, Fort Detrick, MD (US)

(21) Appl. No.: 18/628,287

(22) Filed: Apr. 5, 2024

Related U.S. Application Data
(60) Provisional application No. 63/518,724, filed on Aug. 10, 2023.

(51) Int. Cl. A61B 5/00; A61B 5/02; A61B 5/021; A61B 17/00; A61B 17/132; A61B 17/135; A61M 5/142; G16H 20/17; G16H 20/30; G16H 40/63

(52) U.S. Cl. CPC A61B 5/4839; A61B 5/02042; A61B 5/021; A61B 5/7275; A61B 17/1325; A61B 17/1355; A61M 5/14228; G16H 20/17; G16H 20/30; G16H 40/63; A61B 2017/00022; A61M 2205/10; A61M 2205/3303; A61M 2205/3327; A61M 2205/3334; A61M 2205/50; A61M 2230/005; A61M 2230/04

(57) ABSTRACT

Methods, devices, systems, and computer-readable media are described for providing adaptive and closed loop hemorrhagic shock resuscitation. A computing device may cause activation of an automated tourniquet configured to impede extremity bleeding. The computing device may additionally cause activation of an adaptive resuscitation controller by determining a desired mean arterial pressure setpoint and causing, based on the desired mean arterial pressure setpoint, the adaptive resuscitation controller to infuse fluids into the patient. The computing device may then monitor component pressure measurements of the automated tourniquet and blood pressure parameters of the patient. The computing device may then, based on the component pressure measurements, cause modification of one or more operating parameters of the automated tourniquet. The computing device may additionally and/or alternatively, based on comparing the blood pressure parameters to the desired mean arterial pressure setpoint, cause the adaptive resuscitation controller to infuse fluids into the patient.

### CROSS REFERENCE TO RELATED APPLICATIONS

This application is a non-provisional of U.S. Application No. 63/518,724, entitled “Evaluation of Adaptive Closed Loop Hemorrhagic Shock Resuscitation Controllers,” and filed Aug. 10, 2023. The contents of the above listed application is expressly incorporated herein by reference in its entirety for any and all non-limiting purposes.

### FIELD OF USE

Aspects of the disclosure relate generally to systems for hemorrhagic shock resuscitation. More particularly, aspects described herein relate to an adaptive and closed loop hemorrhagic shock resuscitation controller device implementing various algorithms to control an adaptive resuscitation controller and other devices, such as an automated tourniquet.

### BACKGROUND

Given the evolving nature of patient care and military conflicts, there is an ever-present need for processes that automate, streamline, and/or otherwise augment the delivery of medical care in both emergency and combat trauma environments. Along those lines, various simple devices for automated patient care have been devised. For example, an autonomous device might be able to take care of providing ventilation to a patient under certain conditions, offloading the responsibility of this task away from a human medical provider (who can then be tasked with a different role, improving patient care overall). Such devices are generally closed-loop devices: they receive some simplistic input data (e.g., whether the patient is breathing on their own) and make a care decision based on that data (e.g., whether to aid in ventilation). This simplicity has various advantages: it keeps the devices easy to use, relatively predictable and reliable, repairable, and cheap.

With that said, managing multiple automated devices simultaneously, all potentially competing with one another, can be difficult, particularly in an environment where human medical providers might also be providing medical care at the same time. As an example of this problem, each of a variety of different automated patient care devices might be capable of executing independently but might not be aware of a patient's overall status. In turn, the myopic perspective of these devices (and the simple fact that they are not aware of the existence of other devices) could cause devices to provide contradictory care. It is often the case in perioperative settings and intensive care units that, when a patient requires multiple interventions simultaneously, a clinician will prioritize one line of treatment at the expense of another—that said, autonomous systems cannot make similar decisions, as they do not have such insight into the overall status of a patient. This can have undesirable consequences for a patient: after all, in extreme situations, two different caregiving devices locked in inadvertent competition for one another might end up harming, rather than helping, the patient.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below.

Aspects described herein relate to an adaptive closed loop hemorrhagic shock resuscitation controller. This process implements a Supervisory Algorithm for Casualty Management (SACM) device that manages decisions and interplay between various automated systems, such as those designed for the management of hemorrhage control (e.g., an automated tourniquet system, such as an automated tightening tourniquet device) and resuscitation (e.g., an adaptive resuscitation controller). This system advantageously allows the SACM device to implement different approaches to prioritizing patient care by, for example, carefully controlling the infusion of fluids into a patient in view of other factors, such as the application of a tourniquet. This system also monitors physiological inputs for both systems and synchronizes the systems in view of an overall patient status, in effect preventing the systems from conflicting and improving patient care overall. The SACM's process is generally performed in two stages: an active intervention stage (whereby devices are activated and are implemented to some form of a baseline-such as activating a tourniquet and causing it to tighten and try to impede bleeding, or activating an adaptive resuscitation controller and causing it to begin to monitor patient vitals and deliver fluids), and then a patient monitoring stage (whereby the SACM device continually monitors the status of various devices and the patient and, as needed, activates and/or otherwise modifies the operating parameters of the devices to maintain patient care). In this way, the SACM may use optimized algorithms to provide fluid infusions at an optimal rate while simultaneously monitoring and controlling different (albeit related) patient care priorities, such as the inhibition of hemorrhage.

More particularly, a computing device, such as the aforementioned SACM device, may be configured to provide adaptive and closed loop hemorrhagic shock resuscitation using an automated tourniquet and an adaptive resuscitation controller. The computing device may cause activation of the automated tourniquet by causing the automated tourniquet to increase pressure of a component of the automated tourniquet to impede extremity bleeding of a patient. The computing device may then cause causing activation of the adaptive resuscitation controller by determining a desired mean arterial pressure setpoint and causing, based on the desired mean arterial pressure setpoint, the adaptive resuscitation controller to infuse a first quantity of fluids into the patient. In either or both cases, causing activation of either device may comprise deactivating an automatic functioning of the device, in effect ceding control of operations of those devices to the computing device. The computing device may monitor component pressure measurements corresponding to the component of the automated tourniquet and, based on the component pressure measurements, cause modification of one or more operating parameters of the automated tourniquet. The computing device may also monitor blood pressure parameters of the patient and, based on comparing the blood pressure parameters to the desired mean arterial pressure setpoint, cause the adaptive resuscitation controller to infuse a second quantity of fluids into the patient. The monitoring of both the automated tourniquet and the adaptive resuscitation controller (and any reactions thereto, such as modifying the operating parameters of either) may be performed at the same time (e.g., in parallel). Moreover, various actions by the computing device (e.g., tightening the automated tourniquet) may be performed based on a wide variety of data, such as the blood pressure parameters of the patient, rather than just data typically available to the automated tourniquet.

Aspects described herein thereby allow the computing device to react to circumstances where one or more devices are not providing an adequate level of care. For example, as part of causing modification of the one or more operating parameters of the automated tourniquet, the computing device may detect air pressure oscillations in the component and then increase a volume of air in the component. In this way, if (for instance) the automated tourniquet loosens or otherwise stops adequately impeding hemorrhage, the computing device may react and provide necessary patient care. As another example, the computing device may detect, based on the component pressure measurements, hemorrhage associated with a first appendage of the patient, even in circumstances where the automated tourniquet is configured to impede extremity bleeding of a second appendage of the patient. In this circumstance (e.g., where tightening of the tourniquet would not stop the newly-discovered hemorrhage), the computing device may take various steps, such as causing the adaptive resuscitation controller to cease infusion of fluids.

A computing device implementing such controller may infuse a quantity of fluid into the patient based on a desired mean arterial pressure setpoint. This process may entail determining intermediate mean arterial pressure setpoints for the adaptive resuscitation controller. As will be described further below, this approach has numerous advantages, including adapting to patient changes in blood pressure in response to fluid as well as potentially avoiding providing too much fluid to a patient. For example, the computing device may determine, based on the desired mean arterial pressure setpoint, a plurality of different intermediate mean arterial pressure setpoints. In turn, causing the adaptive resuscitation controller to infuse the first quantity of fluids into the patient may be based on a first intermediate mean arterial pressure setpoint of the plurality of different intermediate mean arterial pressure setpoints, and causing the adaptive resuscitation controller to infuse the additional quantity/quantities of fluids into the patient may be based on a an intermediate mean arterial pressure setpoint of the plurality of different intermediate mean arterial pressure setpoints.

The infusion of fluids by the adaptive resuscitation controller may be based, in whole or in part, on a correction factor. The computing device may determine a correction factor based on a difference between a predicted time of an increase in mean arterial pressure and an actual time of an increase in mean arterial pressure indicated by the blood pressure parameters of the patient. In turn, the fluids infused into the patient may be based on this correction factor. This, as with the above approach of using intermediate mean arterial pressure setpoints, can aid the adaptive resuscitation controller in providing an appropriate amount of fluids to a patient.

The infusion of fluids may be based on mathematical calculations and/or assumptions. For example, the computing device may determine a volume-pressure relationship between one or more of a total fluid volume associated with the patient or a volume of the first quantity of fluids and at least one of the blood pressure parameters, then determine, based on the volume-pressure relationship, a volume of the additional quantities of fluids. Additionally and/or alternatively, the volume of fluids provided to a patient may be determined, in whole or in part, based on a Dubick constant of approximately 0.337 ml/mmHg/kg.

Broadly, the goal of the infusion of fluids may be to bring the patient to a baseline value, such as the aforementioned mean arterial pressure point. In turn, the computing device may, based on determining that the blood pressure parameters are within 0.5% of the desired mean arterial pressure setpoint, cause the adaptive resuscitation controller to cease infusion of fluids.

These features, along with many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIG. 1 depicts an illustrative system comprising a supervisory algorithm for casualty management device, an adaptive resuscitation controller device, and an automated tightening tourniquet device.

FIG. 2 depicts an illustrative two-stage process for adaptive closed loop hemorrhagic shock resuscitation, including an active intervention stage and a patient monitoring stage.

FIG. 3 is a flow chart depicting steps for adaptive closed loop hemorrhagic shock resuscitation using an automated tourniquet and an adaptive resuscitation controller.

FIG. 4 depicts an illustrative adaptive resuscitation controller device implemented in interacting with a physiology simulator of cardiovascular function.

FIG. 5 depicts an illustrative Physio Vessel test platform.

FIG. 6 depicts an illustrative Hardware-In-Loop Testbed for Resuscitation Controller platform for testing an adaptive resuscitation controller for hemorrhagic shock.

FIG. 7A shows a chart showing mean pressure and flow rate over time for a scenario managed by an adaptive resuscitation controller where mean arterial pressure begins at 45 mmHg with an aggressive hemorrhage rate that initially slows and later accelerates.

FIG. 7B shows a chart showing mean pressure and flow rate over time for a scenario managed by an adaptive resuscitation controller where mean arterial pressure begins at 65 mmHg with an aggressive hemorrhage rate that slows.

FIG. 7C shows a chart comparing scores for different adaptive resuscitation controllers.

FIG. 7D depicts a chart showing mean arterial pressure over time for a study for whole blood resuscitation and a two-hour hold in swine, as managed by an adaptive resuscitation controller.

FIG. 8A comprises charts showing whole blood infusion rates comparing baseline and with a 0.5× correction factor.

FIG. 8B comprises charts showing crystalloid infusion rates comparing baseline and with whole blood infusion rates at a 2× correction factor.

FIG. 8C comprises charts showing infusion rates for a whole blood Physio Vessel with a 240 mL/min hemorrhage and a 120 mL/min hemorrhage.

FIG. 8D comprises charts showing infusion rates for a crystalloid Physio Vessel with a 240 mL/min hemorrhage and a 360 mL/min hemorrhage.

FIG. 9A depicts charts showing distal mean arterial pressure, tourniquet pressure, central mean arterial pressure, infusion rate, and fluid balance over time for a first testing scenario.

FIG. 9B depicts charts showing distal mean arterial pressure, tourniquet pressure, central mean arterial pressure, infusion rate, and fluid balance over time for a second testing scenario.

FIG. 9C depicts charts showing distal mean arterial pressure, tourniquet pressure, central mean arterial pressure, infusion rate, and fluid balance over time for a third testing scenario.

FIG. 9D depicts charts showing distal mean arterial pressure, tourniquet pressure, central mean arterial pressure, infusion rate, and fluid balance over time for a fourth testing scenario.

FIG. 9E depicts charts showing distal mean arterial pressure, tourniquet pressure, central mean arterial pressure, infusion rate, and fluid balance over time for a fifth testing scenario.

FIG. 10A comprises charts showing various comparisons of resuscitation paces for different types of controllers.

FIG. 10B shows charts indicating mean pressure and flow rate over time, as well as comparisons of various controllers with respect to target pressure, for a first scenario.

FIG. 10C shows charts indicating mean pressure and flow rate over time, as well as comparisons of various controllers with respect to target pressure, for a second scenario.

FIG. 10D shows charts indicating mean pressure and flow rate over time, as well as comparisons of various controllers with respect to target pressure, for a third scenario.

FIG. 10E shows charts indicating mean pressure and flow rate over time, as well as comparisons of various controllers with respect to target pressure, for a fourth scenario.

FIG. 10F comprises charts comparing various controllers in terms of overshoot, variable infusion, end-state divergence, and overall score.

FIG. 11 depicts an example of a computing device that may be used in implementing one or more aspects of the disclosure in accordance with one or more illustrative aspects discussed herein.

### DETAILED DESCRIPTION

In the following description of the various embodiments, reference is made to the accompanying drawings, which form a part hereof, and in which is shown by way of illustration various embodiments in which aspects of the disclosure may be practiced. It is to be understood that other embodiments may be utilized and structural and functional modifications may be made without departing from the scope of the present disclosure. Aspects of the disclosure are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof.

As an introduction, autonomous and semi-autonomous medical systems may be able to rapidly execute adjustments in care in response to a patient's needs, thereby possibly providing a significant benefit to patient care. Indeed, those rapid adjustments are particularly critical for patient management in the perioperative and intensive care setting. Such devices include, but not limited to, ventilator management devices, fluid resuscitation devices, pain control and sedation devices, and the like. With that said, such devices often operate independently and in a closed-loop manner, their course of care can contradict. While human caregivers often have the ability to, for example, prioritize a single form of care over others, such closed-loop devices lack this functionality. This is particularly the case over time: while devices might be fairly competent at performing simple tasks (e.g., a ventilator management device might start up and begin providing ventilation reasonably well), over time and with the addition of multiple devices running at different times, the complexity becomes significantly more difficult, and the likelihood of device conflict becomes higher. For example, in some circumstances, a sedation-providing device might still try to sedate a patient when other forms of care (e.g., providing a tourniquet) are significantly more important and somewhat contradictory.

Aspects described herein use a Supervisory Algorithm for Casualty Management (SACM) to manage various devices and ensure a consistent and careful application of care, particularly with respect to the provision of infused fluids to a patient. Specifically, aspects disclosed herein describe a process whereby an Adaptive Resuscitation Controller (ARC) device can be used to infuse fluids for a user while an Automated Tightening Tourniquet (aTKT) device is simultaneously used to control patient hemorrhage. The SACM can use particular algorithms (detailed further below) to carefully control this fluid infusion, thereby optimizing patient care. In this manner, both devices can work quickly and in a complementary manner, rather than in a contradictory manner. Broadly, this process is effectuated through a two-stage process. First, the SACM activates each device in a manner which allows the device to bring the patient to (as best as practicable) a baseline: for example, a tourniquet might tighten to impede hemorrhage, whereas an adaptive resuscitation controller might, based on some algorithm, mathematical relationship, or similar calculation process, infuse fluids to, as best as practical, begin to raise the mean arterial pressure of a patient. Then, the SACM might then monitor measurements and selectively activate and/or modify aspects of either device, in effect working to maintain the baseline. The SACM can continually use the aforementioned algorithm, mathematical relationship, or similar calculation process to provide such care, generally ensuring that a patient is brought to a desired baseline (e.g., a desired mean arterial pressure) in a desirable way (e.g., quickly, without excessive overshoot).

As suggested above, one particularly beneficial aspect of the aspects described above is that the processes described herein can implement a variety of algorithmic approaches for maintaining patient care. Of note, the approach described herein can use at least four different approaches to infusing fluids via the adaptive resuscitation controller: Absolute-ARC (A-ARC), which relates total fluid volume in the system to the pressure (p=m*V+P₀), Relative-ARC (R-ARC), which relates a change in volume in the system to a change in pressure (ΔP=m*ΔV+0), Dubick-o-Matic ARC (D-ARC), which relies on the Dubick constant (that is, approximately 0.337 ml/mmHg/kg), and a Dual Fuzzy Logic (DFL) approach. Such approaches can be taken with respect to a linear regression. These approaches are discussed at length below via test data on a test platform.

Aspects described herein improve the functioning of computers at least because the improvements serve to improve the way in which different devices (e.g., automated tourniquet devices, adaptive resuscitation controller devices, ventilator management devices, fluid resuscitation devices, pain control and sedation devices, and the like) operate, particularly where those devices might provide contradictory care. Such devices are often closed-loop systems with incredibly rudimentary functionalities. While such simplicity has advantages (case of use, case of repair, cost), it can also mean that multiple such devices can provide contradictory care for a patient, even when monitored by a human care provider. The aspects described herein remedy these and other issues by creating a system with a supervisory device that, in effect, acts as a proverbial traffic cop for the care provided by various devices. The result of such computer improvements is fundamentally better and more consistent patient care.

### ARC Device

Discussion will begin with a broad overview of an illustrative system for implementing aspects described herein.

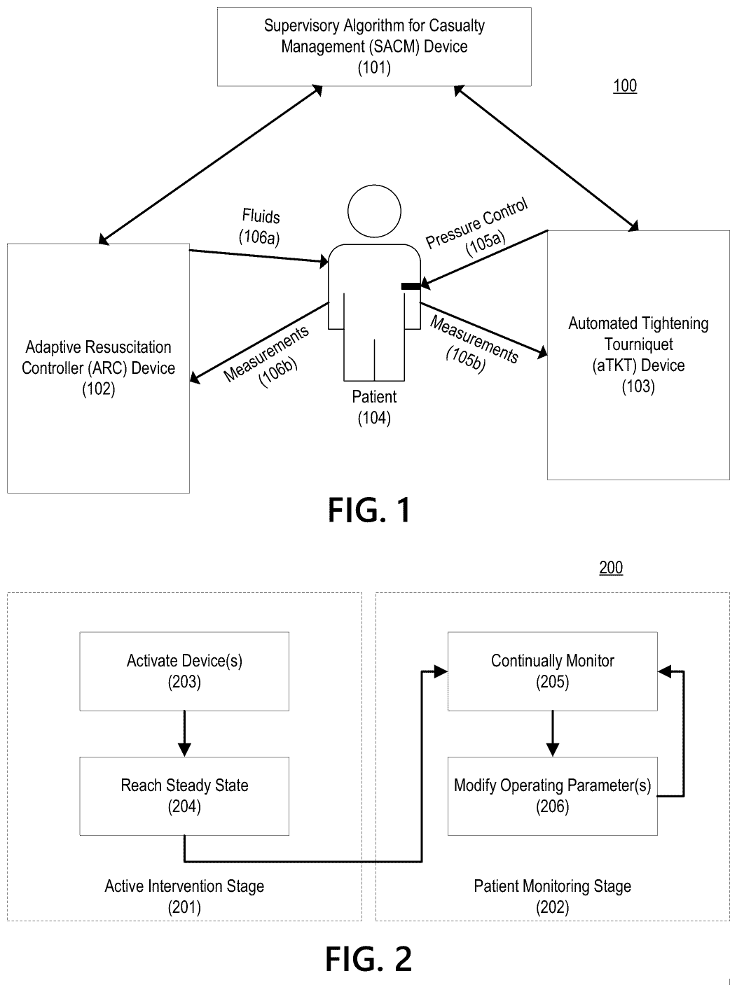

FIG. 1 depicts an illustrative system 100 comprising a Supervisory Algorithm for Casualty Management (SACM) device 101, an Adaptive Resuscitation Controller (ARC) device 102, an Automated Tightening Tourniquet (aTKT) device 103. All such devices are shown with respect to a patient 104. All of these devices may comprise computing devices, such as a computing device comprising one or more processors and memory storing instructions that, when executed by one or more processors, cause various steps to be performed. The ARC device 102 may be configured to deliver fluids to the patient 104 (as represented by arrow 106a) and receive measurements of the patient 104 (as represented by arrow 106b). The aTKT device 103 may be configured to control the pressure of one or more tourniquets (as represented by arrow 105a) and receive measurements of the patient 104 (as represented by arrow 105b).

The SACM device 101 may be configured to manage care of the patient 104 by controlling the functions of the ARC device 102 and the aTKT device 103. Because the patient 104 is being treated by both the ARC device 102 and the aTKT device 103 and are not the same device or communicatively coupled, there may be a possibility that the two devices provide contradictory care to the patient 104. To remedy this issue, the SACM device 101 may be configured to monitor and/or control operations of the ARC device 102 and/or the aTKT device 103 at the same time. As a simple example, the SACM device may be configured to prevent the aTKT device 103 from applying additional pressure to a tourniquet in a manner inconsistent with the provision of fluids by the ARC device 102.

The SACM device 101 may control various devices (such as the ARC device 102 and/or the aTKT device 103) using instructions transmitted to those devices, and the SACM device 101 may receive measurements from those devices using a similar process. For example, one or more of the devices may be connected to one or more networks, such that the devices may communicate (e.g., the SACM device 101 may transmit instructions to the ARC device 102 and/or receive measurements from the aTKT device 103) via the network. With that said, the devices may be communicatively coupled in a variety of ways. For example, the ARC device 102 may connect to the SACM device 101 via a simple Universal Serial Bus (USB) cable or the like.

### Processes

Discussion will now turn to processes which may be performed by devices depicted in FIG. 1, such as the SACM device 101, the ARC device 102, and/or the aTKT device 103.

FIG. 2 depicts an illustrative two-stage process for adaptive closed loop hemorrhagic shock resuscitation, including an active intervention stage 201 and a patient monitoring stage 202. In the active intervention stage 201, in step 203, a computing device (such as the SACM device 101) may activate one or more devices, such as the ARC device 102 and/or the aTKT device 103. These activated devices may be configured to, upon activation, perform various steps to reach a patient steady state, as reflected in step 204. For example, the ARC device 102 may begin providing fluids, ultimately reaching a desired mean arterial pressure value for the patient. As another example, the aTKT device 103 may be configured to tighten a tourniquet until it has detected that hemorrhaging of a given appendage has been sufficiently impeded or stopped.

In the patient monitoring stage 202, the computing device (e.g., the SACM device 101) may perform various steps to maintain the patient steady state established in step 204. This may include, for example, ensuring that various devices do not fail to operate, that the devices continue to provide any ongoing care necessary for the patient, and the like. In step 205, the computing device may continually monitor measurements taken by the devices (e.g., the measurements taken by the ARC device 102 and/or the aTKT device 103). For example, the computing device may monitor patient blood pressure, the pressure of various tourniquets applied by the aTKT device 103, and the like. Then, in step 206, the computing device may modify operating parameters based on the monitoring performed in step 205. For instance, if the pressure of various tourniquets applied by the aTKT device 103 suggests that a particular tourniquet has become loose, then instructions may be transmitted from the computing device to the aTKT device 103 that cause the aTKT device 103 to increase the tightness of the tourniquet. As another example, if the blood pressure of a patient drops below a threshold, then instructions may be transmitted from the computing device to the ARC device 102 that cause the device to provide additional fluids to the patient.

In both stages, the SACM device 101 may implement an algorithm, mathematical calculation, mathematical constant, or similar processing to control the flow of fluids to a patient. For example, the SACM device 101 may collect data from a variety of data sources (e.g., the ARC device 102, the aTKT device 103, and/or other sources) and make decisions as to how to control the flow rate of an infused fluid (e.g., whole blood, crystalloid fluid) based on that data. In this manner, the SACM device 101 can optimize patient care in a manner that not only avoids device conflict, but also in a manner that potentially expedites bringing a patient to a desired baseline.

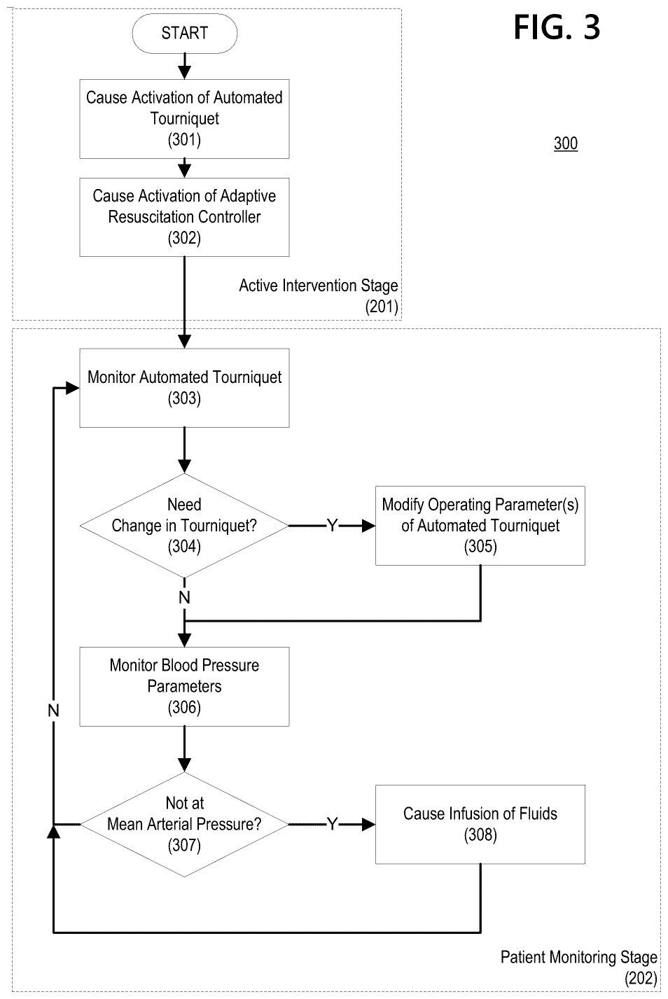

FIG. 3 is a flow chart depicting steps for a method 300 for adaptive closed loop hemorrhagic shock resuscitation using an automated tourniquet (e.g., the aTKT device 103) and an adaptive resuscitation controller (e.g., the ARC device 102). The steps depicted in FIG. 3 may be performed by one or more computing devices, such as the SACM device 101. A computing device may comprise one or more processors and memory storing instructions that, when executed by the one or more processors, cause performance of one or more of the steps of FIG. 3. One or more non-transitory computer-readable media may store instructions that, when executed, cause a computing device to perform one or more of the steps of FIG. 3. The steps shown in FIG. 3 are illustrative, and might be rearranged or otherwise modified if desired. For instance, various aspects of the later steps of FIG. 3 might be rearranged to account for additional and/or different devices (instead of, as depicted in FIG. 3, an automated tourniquet and/or an adaptive resuscitation controller). Similar to FIG. 2, FIG. 3 begins with the active intervention stage 201 and proceeds to the patient monitoring stage 202.

In step 301, which begins the active intervention stage 201, the computing device may cause activation of an automated tourniquet, such as the aTKT device 103. This step may comprise transmitting instructions to the automated tourniquet and thereby causing it to activate and, as applicable, cause the automated tourniquet to impede extremity bleeding of a patient. For example, the computing device may cause activation of the automated tourniquet by causing the automated tourniquet to increase pressure of a component of the automated tourniquet to impede extremity bleeding of a patient.

In step 302, the computing device may cause activation of an adaptive resuscitation controller, such as the ARC device 102. This process may comprise causing the ARC device 102 to perform steps to bring the patient to a determined desired mean arterial pressure setpoint. For example, the computing device may cause activation of the adaptive resuscitation controller by determining a desired mean arterial pressure setpoint and causing, based on the desired mean arterial pressure setpoint, the adaptive resuscitation controller to infuse a first quantity of fluids into the patient.

As part of determining the desired mean arterial pressure setpoint, the computing device may determine a variety of intermediate mean arterial pressure setpoints. Recognizing that a patient might require an uncertain (and potentially changing) volume of fluids to reach a desired mean arterial pressure, the computing device may determine certain setpoints (e.g., checkpoints) to reach and evaluate how to properly reach (and not undershoot or exceed) the desired mean arterial pressure setpoint. For example, the computing device may determine, based on the desired mean arterial pressure setpoint, a plurality of different intermediate mean arterial pressure setpoints. For instance, for a desired mean arterial pressure setpoint of 65 mmHg and a beginning arterial pressure of 45 mmHg, the computing device may determine different intermediate mean arterial pressure setpoints at increments of 5 mmHg (that is, 50 mmHg, 55 mmHg, 60 mmHg, and finally 65 mmHg). In this manner, the computing device might first provide a first quantity of fluids (e.g., a first bolus of 100 mL over a minute), and then later, based on subsequent measurements, provide a different quantity of fluids (e.g., 90 mL, 110 mL, or the like).

Both step 301 and/or step 302 may involve disabling some or all aspects of the automated tourniquet and/or the adaptive resuscitation controller. Left alone, such devices might operate automatically and without waiting for instructions from the computing device, which might be undesirable in circumstances where the computing device wants to control when (if at all) the devices are operating. As such, the devices might be switched into a manual mode or otherwise configured to activate only upon instruction from the computing device. For example, the computing device may deactivate an automatic functioning of the automated tourniquet and/or may deactivate an automatic functioning of the adaptive resuscitation controller.

After step 302, a patient may have been brought to a relatively safe condition. For example, the automated tourniquet may be (to the extent practicable) impeding hemorrhaging, and the adaptive resuscitation controller may have brought the patient to a desired mean arterial pressure setpoint (or, more practically, might be actively working to bring the patient to that setpoint through the infusion of fluids over some time period).

In step 303, which begins the patient monitoring stage 202, the computing device may monitor the automated tourniquet. This process may comprise monitoring the tightness of (e.g., the pressure of the air inside a bladder of) the tourniquet(s) of the automated tourniquet. For example, the computing device may monitor component pressure measurements corresponding to the component of the automated tourniquet.

Then, in step 304, the computing device may evaluate data collected during the monitoring process of step 303 to determine whether the automated tourniquet should be modified. For instance, a large drop of air (e.g., 33%) might suggest that the tourniquet has become undesirably loose (and/or a mechanical failure), requiring operational changes. Such modification might comprise, for instance, changing operating parameters of the automated tourniquet to tighten, untighten, or otherwise tweak the effectiveness of one or more tourniquets. If there is a needed change in the tourniquet, the method 300 proceeds to step 305. Otherwise, the method 300 proceeds to step 306.

In step 305, and if the computing device determined that a change in the tourniquet was needed, the computing device may modify one or more operating parameters of the automated tourniquet. For example, the computing device may cause, based on the component pressure measurements, modification of one or more operating parameters of the automated tourniquet. As suggested above, this may include tightening, loosening, and/or otherwise modifying one or more components of the tourniquet. Such modification might be further based on blood pressure parameters collected via other devices, such as the ARC device 102. In other words, the tourniquet might be modified based on processing, by the computing device, of a wide variety of data available, not merely the data available via the aTKT device 103.

As an example of step 303, step 304, and step 305, the computing device may detect air pressure oscillations in a component of the automated tourniquet. This may be indicative of a variety of undesirable circumstances, such as the possibility that the tourniquet is not sufficiently tight on an appendage of the patient. In such a circumstance, the computing device might tighten the component by, for instance, increasing a volume of air in the component.

In step 306, the computing device may monitor blood pressure parameters of a patient. Step 306 may involve monitoring a broad variety of aspects of a patient, such as their heart rate, blood pressure, the components of their blood, their heart rate, or the like. Indeed, FIG. 3 focuses on blood pressure merely because it provides a useful example of how the adaptive resuscitation controller might respond to such a change.

In step 307, the computing device may determine whether the blood pressure of the patient (as measured as part of step 306) is at or around the mean arterial pressure. If the blood pressure of the patient is at or around the mean arterial pressure, then the computing device might not need to make a change, and the method 300 might return back to step 303. With that said, if the blood pressure of the patient is not at or around the mean arterial pressure, the method 300 may proceed to step 308.

In step 308, the computing device may cause an infusion of fluids to the patient. For example, the computing device may cause, based on comparing the blood pressure parameters to the desired mean arterial pressure setpoint, the adaptive resuscitation controller to infuse a second quantity of fluids into the patient. The fluids may be infused based on the blood pressure parameters (e.g., as measured via the ARC device 102), the component pressure measurements (e.g., as measured by the aTKT device 103), or the like. In other words, the fluids may be infused based on processing, by the computing device, of a wide variety of data available, not merely the data available via the ARC device 102.

The fluids infused might be based on a correction factor. The correction factor might be based on differences between a predicted time of mean arterial pressure change of a patient and the actual time of an increase in the mean arterial pressure of a patient. Stated differently, the correction factor might account for the fact that the patient's mean arterial pressure might be responding more slowly or more quickly to infused fluids than expected, such that a correction in the quantity of fluids provided to the patient may be necessary. For example, the computing device may determine a correction factor based on a difference between a predicted time of an increase in mean arterial pressure and an actual time of an increase in mean arterial pressure indicated by the blood pressure parameters of the patient, and additional quantities (e.g., a second quantity) of fluids might be based on the correction factor.

Mathematically, the correction factor for a particular time period might be calculated as a function of previous correction factors, a difference in actual and predicted times in which mean arterial pressure was reached, and using a scaling factor (modifiable by an administrator to change the speed of change of a flow rate) as follows:

$$Correction {Factor}_{n + 1} = Correction {Factor}_{n} ⋆ \frac{{t}_{actual}}{{t}_{predicted}} ⋆ Scaling Factor$$

The fluids infused in both step 302 and 308 might be based on a volume-pressure relationship. The computing device may determine a volume-pressure relationship in at least two ways. On one hand, the volume-pressure relationship may be derived by comparing a total fluid volume associated with a patient with at least one blood pressure parameter. This may yield a total volume-pressure relationship. On the other hand, the volume-pressure relationship may be derived by comparing a volume of a quantity of fluids provided to a patient at a particular time with at least one blood pressure parameter associated with that particular time. This may yield a temporally-based volume-pressure relationship. Either such measurement may be used to determine a quantity of fluids to provide to a patient. In this manner, the computing device might calculate how much fluid to provide to a patient to achieve a desired mean arterial pressure, thereby (to the extent possible) avoiding providing too much or too little fluid at any given time.

Additionally and/or alternatively, the fluids infused in both step 302 and step 308 might be based on a constant, such as an assumed relationship between blood pressure parameters to fluid infusion. For example, the computing device may determine, based on a Dubick constant of approximately 0.337 ml/mmHg/kg, a volume of a quantity of fluids.

In the event that the computing device is providing fluids as part of a plurality of intermediate mean arterial pressure setpoints, the computing device might provide a different quantity of fluids in step 308 as it did in step 302. For example, the computing device might cause the adaptive resuscitation controller to provide a first quantity of fluids based on a first intermediate mean arterial pressure setpoint of the plurality of different intermediate mean arterial pressure setpoints, and then might cause the adaptive resuscitation controller to provide a second quantity of fluids based on a second intermediate mean arterial pressure setpoint of the plurality of different intermediate mean arterial pressure setpoints.

As part of step 308, the computing device may cease infusion of fluids. This may be because a desired mean arterial pressure has been reached. For example, the computing device may, based on determining that the blood pressure parameters are within 0.5% of the desired mean arterial pressure setpoint, cause the adaptive resuscitation controller to cease infusion of fluids. This may additionally and/or alternatively be performed where, for example, data suggests that doing so would be harmful to the patient. For example, the computing device may detect, based on the component pressure measurements, hemorrhage associated with a first appendage of the patient. In such a circumstance, the automated tourniquet may be configured to impede extremity bleeding of a second appendage of the patient. In other words, the automated tourniquet might not be able to, itself, prevent the hemorrhage of the first appendage. In this case, the computing device may cause the adaptive resuscitation controller to cease and/or otherwise modify the infusion of fluids.

As indicated by FIG. 3, step 303 through step 308 may be repeated as desired to continually monitor a patient. For example, the steps depicted in the patient monitoring stage 202 of FIG. 3 may be repeated on a periodic basis (e.g., every ten seconds) so as to ensure that each device monitored by the computing device is providing maximal (and non-contradictory) care to a patient.

Though steps of the patient monitoring stage 202 of FIG. 3 are depicted sequentially, they need not be. For example, steps relating to the automated tourniquet (e.g., step 303, step 304, and/or step 305) may be performed at the same time (e.g., in parallel) with the steps relating to the adaptive resuscitation controller (e.g., step 306, step 307, and/or step 308).

Discussion will now turn to a brief explanation of the ARC device 102.

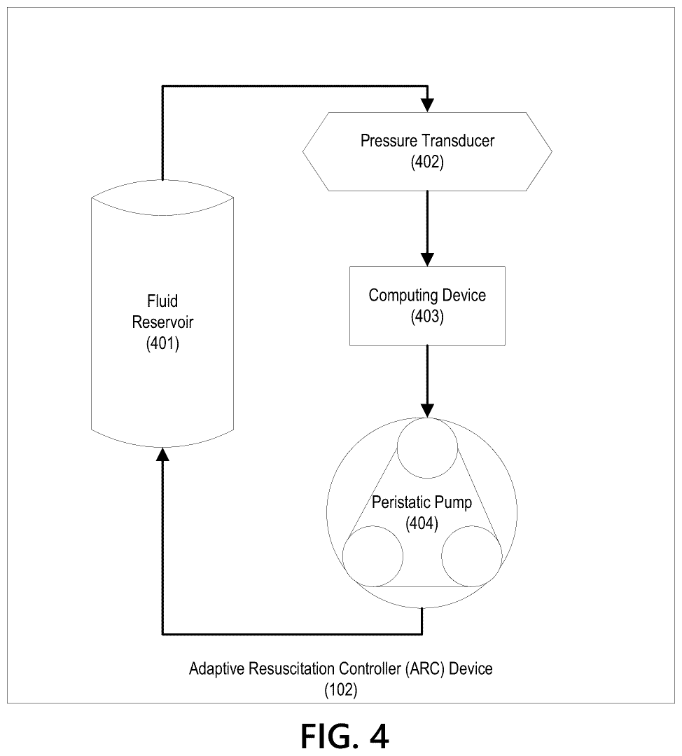

FIG. 4 depicts an illustrative ARC device 102 comprising a fluid reservoir 401, a pressure transducer 402, a computing device 403, and a peristatic pump 404. In the ARC device 102 shown in FIG. 4, a pressure input is sent to the computing device 403 and compared with the desired setpoint, and a flow rate of fluid from the fluid reservoir 401 is adjusted from the setpoint. The computing device 403 is thereby capable of modifying the flow rate by modifying operating parameters of, for example, the pressure transducer 402 and/or the peristatic pump 404.

The ARC device 102 depicted in FIG. 4 may be used to deliver fluids based on instruction(s) received from the SACM device 101. For example, the SACM device 101 may transmit, to the computing device 403 of the ARC device 102, instructions that cause the ARC device 102 to provide a particular quantity of fluids (e.g., 100 mL/min). To implement such instructions, the computing device 403 may control the peristatic pump 404 to pump that volume of liquid from the fluid reservoir 401 and may use the pressure transducer 402 to monitor patient blood pressure and/or the pressure of the fluids exiting the peristatic pump 404.

### Test Environments

Discussion will now turn to various test environments used to develop the above system. The test environments and test data provided below helps underscore, among other things, the unique nature of the innovations described herein, as well as the various ways that the disclosure herein might be implemented (e.g., using different algorithms, different hemorrhage conditions, and the like).

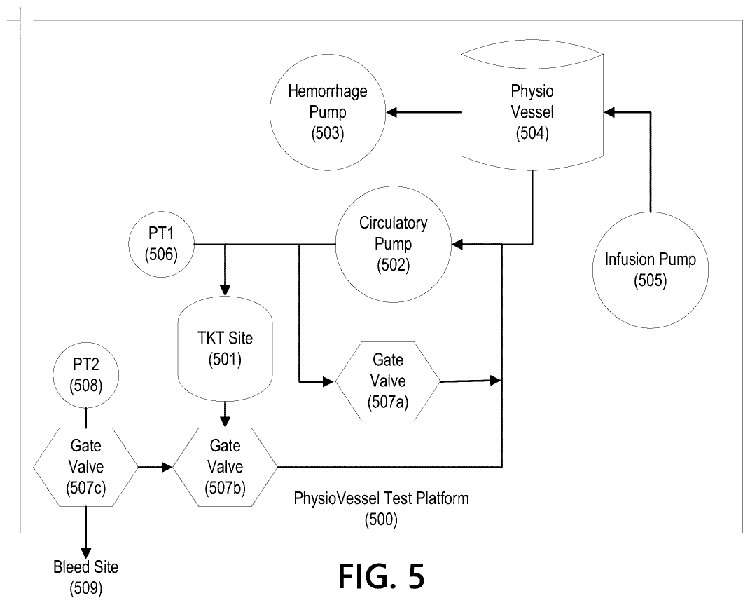

FIG. 5 depicts an illustrative Physio Vessel test platform 500 which can be used to simulate bleeding. In the Physio Vessel test platform 500, various peristaltic pumps (e.g., the circulatory pump 502) and water as the fluid supply may be used to create pulsatile flow through the platform to mimic physiological conditions of a patient. Pressure in the loop may be monitored by a pressure transducer (PT1 506). To enable complete flow occlusion by the tourniquet while permitting continuous flow driven by the circulatory pump 502, a bypass loop may be integrated downstream from PT1 506, with a gate valve 507a allowing tuning to facilitate various circulatory flow rates. A synthetic appendage, such as a phantom arm, might be used to aid in simulation of this system. A t-connector may be used downstream from the synthetic appendage to connect a three-way valve that includes a second pressure transducer (PT2 508) and a bleed site 509. Upstream from the circulatory pump 502 may be a t-connector that integrates a Physio Vessel 504 with a flow loop to provide both volume and pressure to the system. Hydrostatic pressure may be supplied in this system based on the height of the fluid column within the Physio Vessel 504. The relationship between the system volume and this height, and, thus, the pressure within the flow loop, can be precisely controlled to mimic normalized physiological relationships between volume and pressure seen in previous resuscitation studies. Moreover, the hemorrhage pump 503 and the infusion pump 505 may be used to simulate, as desired, internal hemorrhage and infusion of fluid from the adaptive resuscitation controller.

The Physio Vessel test platform 500 may be implemented in at least two ways. First, the Physio Vessel test platform 500 may be implemented as a “whole blood Physio Vessel,” assuming that the infused fluid is whole blood. Second, the PhysioVessel test platform 500 may be implemented as a “crystalloid PhysioVessel,” such that the infused fluid is a crystalloid solution. Various examples of these different PhysioVessels will be described below with respect to different test conditions.

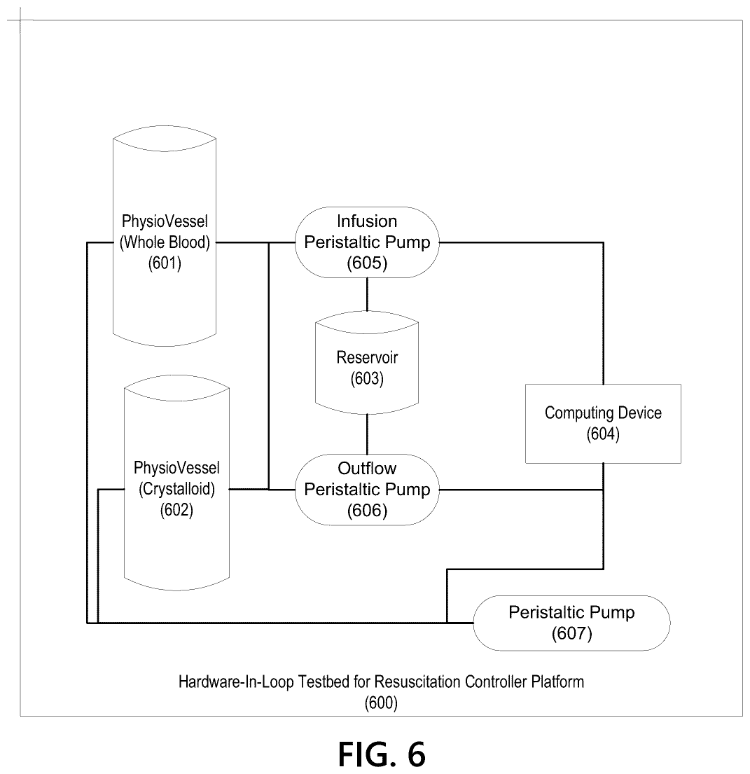

FIG. 6 depicts an illustrative Hardware-In-Loop Testbed for Resuscitation Controller (HATRC) platform 600 depicting two PhysioVessels: a whole blood PhysioVessel 601 and a crystalloid PhysioVessel 602. The whole blood PhysioVessel 601 and the crystalloid Physio Vessel 602 are connected to an infusion peristaltic pump 605 and an outflow peristaltic pump 606, which are both connected to a reservoir 603. The whole blood Physio Vessel 601 and the crystalloid PhysioVessel 602 are also connected to a Peristaltic Pump 607. The infusion peristaltic pump 605, the outflow peristaltic pump 606, and the Peristaltic Pump 607 are all connected to a computing device 604 configured to control the pumps and monitor measurements. The computing device 604 may also be communicatively coupled to various aspects of the Physio Vessels, allowing it to measure, for example, readings from the PT1 506 and/or the PT2 508.

The HATRC platform 600 was the basis for significant testing, including many of the test results provided below. In short, the platform provided a useful tool for emulating human circulatory responses (e.g., blood pressure changes) to, for example, the infusion of fluids, the hemorrhage of those fluids, and the like.

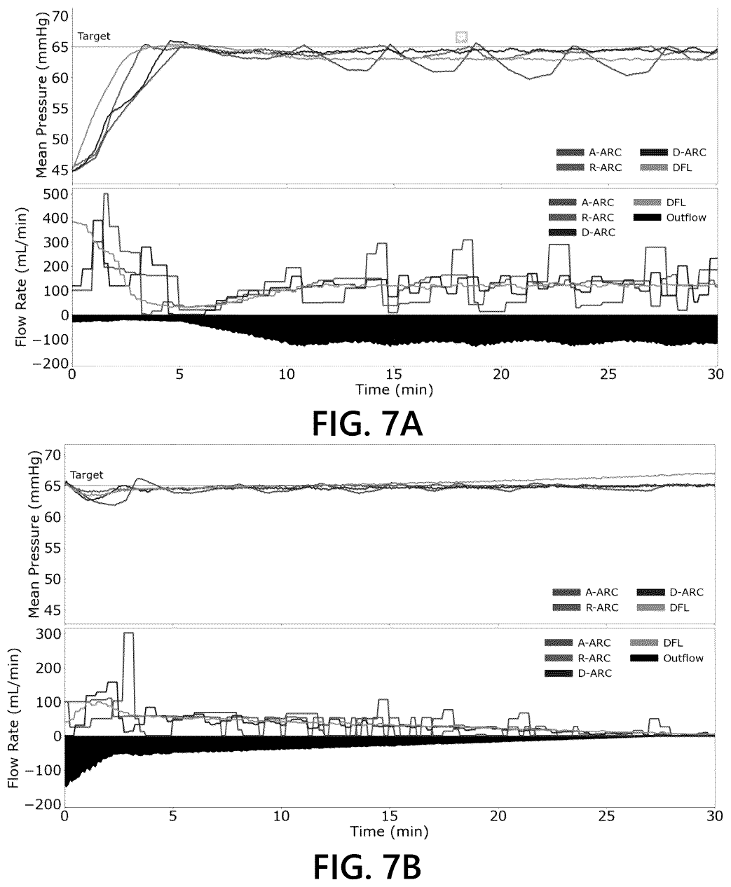

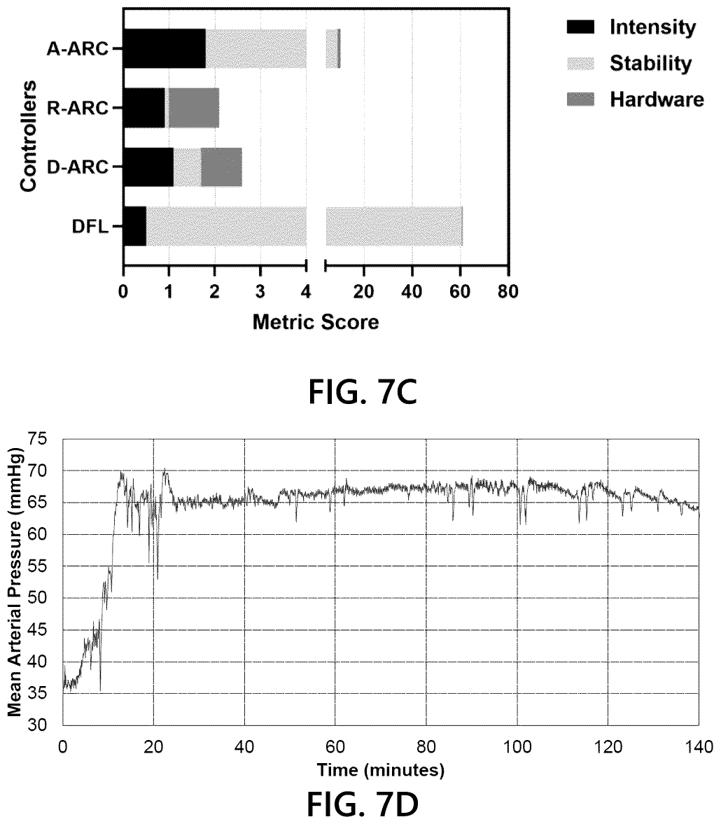

The efficacy of various different algorithms for the ARC device 102 was tested using the aforementioned HATRC platform 600. FIG. 7A shows a chart showing mean pressure and flow rate over time, measured via the HATRC platform 600, for a scenario where mean arterial pressure begins at 45 mmHg with an aggressive hemorrhage rate that initially slows and later accelerates and where the ARC device 102 implements a variety of different algorithms. FIG. 7B is shows a chart showing mean pressure and flow rate over time for the same HATRC platform 600 and for the same algorithms for a scenario where mean arterial pressure begins at 65 mmHg with an aggressive hemorrhage rate that slows. These algorithms include Absolute-ARC (A-ARC), which relates total fluid volume in the system to the pressure (p=m*V+P₀), Relative-ARC (R-ARC), which relates a change in volume in the system to a change in pressure (ΔP=m*ΔV+0), Dubick-o-Matic ARC (D-ARC), which relies on the Dubick constant (that is, approximately 0.337 ml/mmHg/kg), and Dual Fuzzy Logic (DFL) approaches. Such approaches are implemented in this context in a linear regression. Outflow is also shown.

FIG. 7C shows a chart comparing scores for different system controllers (that is, algorithms implemented by the ARC device 102. In this context, lower scores indicate a higher-performing controller, with scores aggregated in terms of intensity, stability, and hardware. This chart generally shows that purpose-built controller designs (e.g., R-ARC, D-ARC) are significantly better than those implementing fuzzy logic, and that in particular the R-ARC algorithm outperformed other designs when tested on the HATRC platform 600.

The efficacy of the R-ARC algorithm as implemented by the ARC device 102 has been shown in a swine study. FIG. 7D depicts a chart showing mean arterial pressure over time for a study for whole blood resuscitation and a two-hour hold in swine using Lactated Ringer's solution. As depicted in this chart, the R-ARC algorithm was able to reach a target MAP (here, 65 mmHg) somewhat quickly, and was able to hold MAP for two hours.

Discussion will now turn to additional testing and results of the ARC device 102, with a particular focus on different Physio Vessels (e.g., the PhysioVessel Test Platform 500, configured to infuse whole blood and/or crystalloid solution).

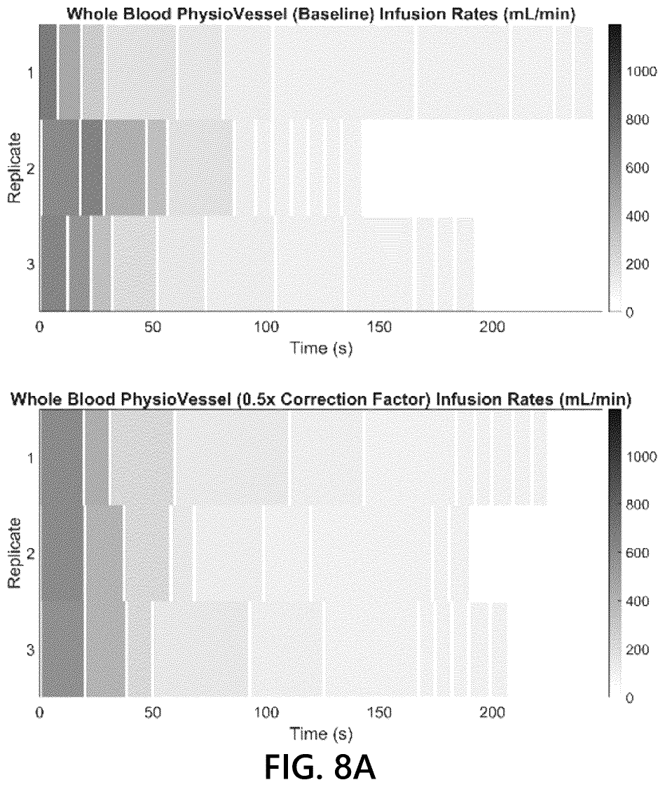

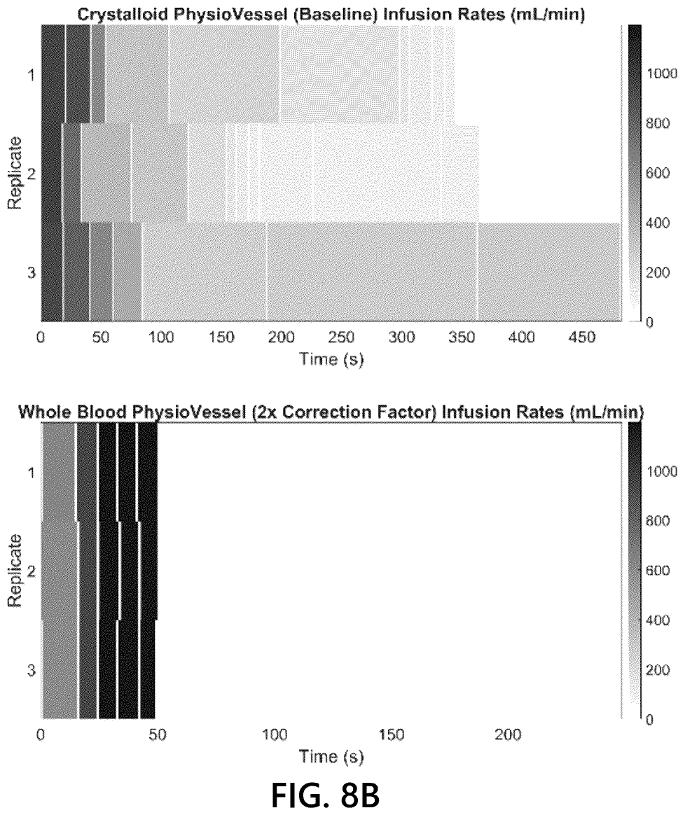

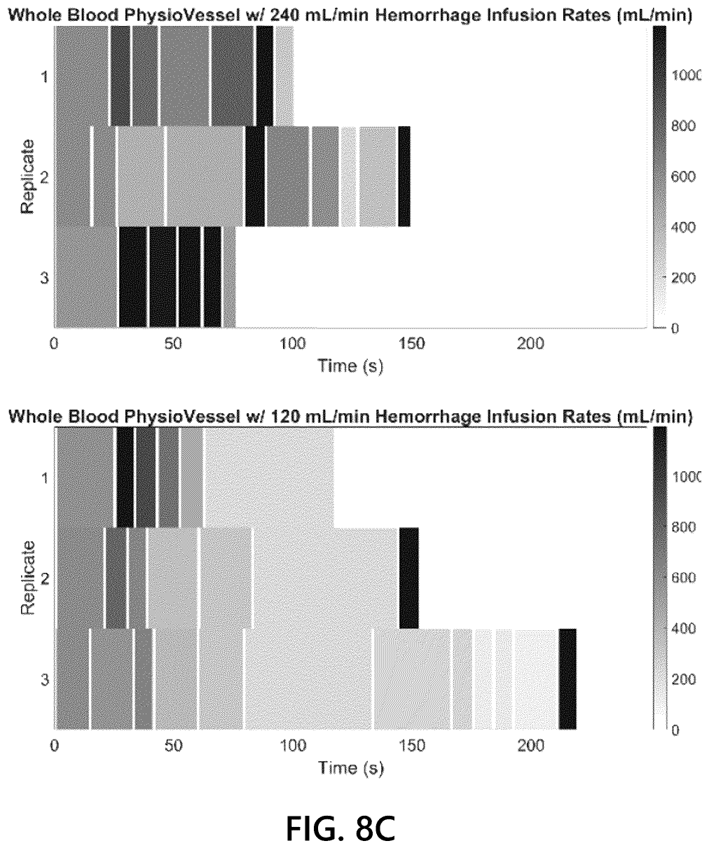

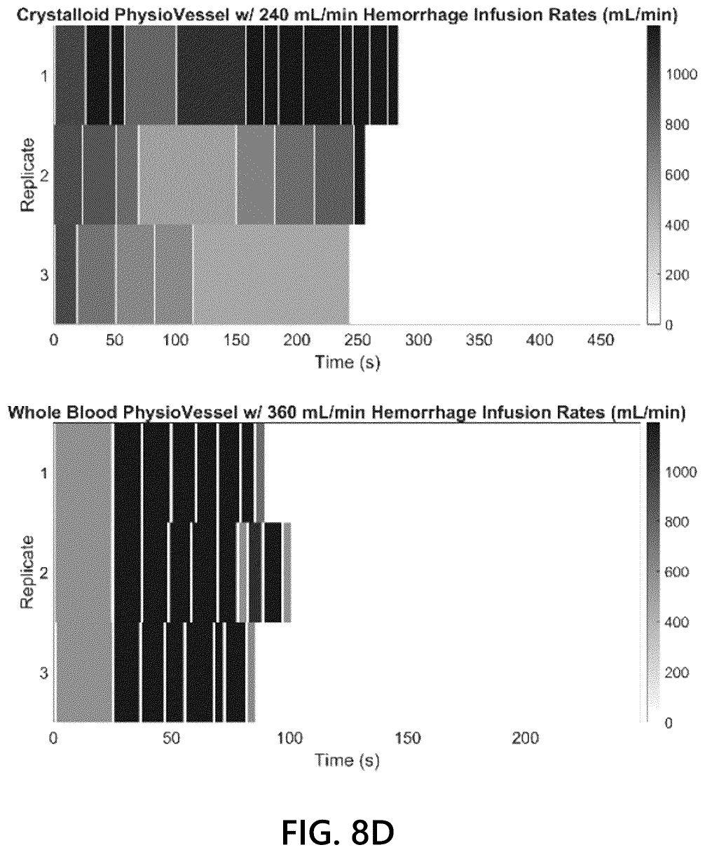

FIG. 8A comprises charts showing whole blood infusion rates comparing baseline and with a 0.5× correction factor. FIG. 8B comprises charts showing crystalloid infusion rates comparing baseline and whole blood infusion rates with a 2× correction factor. These results generally show that, for whole blood, initially high flow rates would reduce to near minimum values as a target setpoint was reached, and that similar results were evident with crystalloid at baseline (although flow rates did not drop as low and required more time to reach the setpoint). When modified, the correction factor changed this quite a bit: for example, when the factor was increased to 2, the flow rate increased and held to the maximum 1200 mL/min almost immediately.

FIG. 8C comprises charts showing infusion rates for a whole blood Physio Vessel with a 240 mL/min hemorrhage and a 120 mL/min hemorrhage. FIG. 8D comprises charts showing infusion rates for a crystalloid Physio Vessel with a 240 mL/min hemorrhage and a 360 mL/min hemorrhage. These results generally show the effect of an internal hemorrhage on the ARC device 102. In these charts, three technical replicates are shown to illustrate how step sizes and flow rate changes varied. Much of the delay in responsiveness between these approaches can be attributed to variability in switching in and out of hemorrhage detection, which was slightly less pronounced for whole blood as compared to crystalloid.

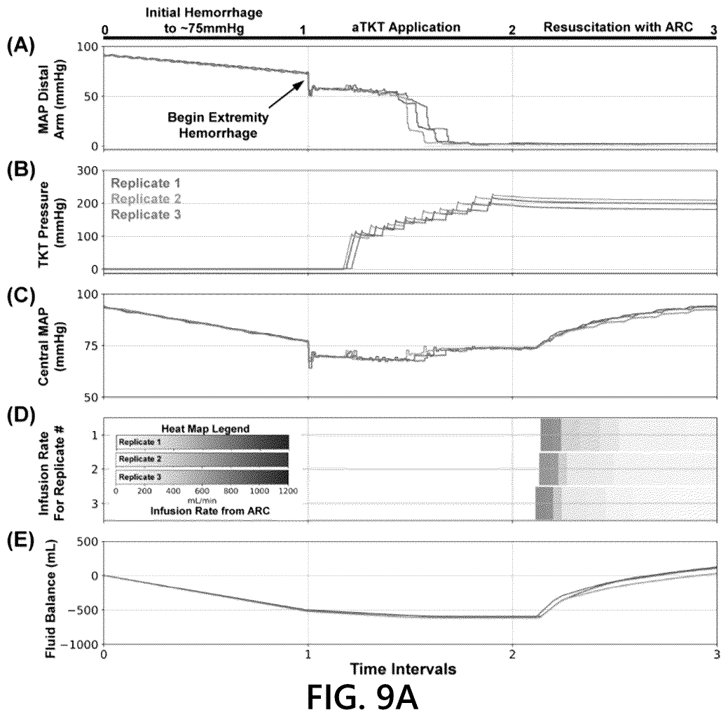

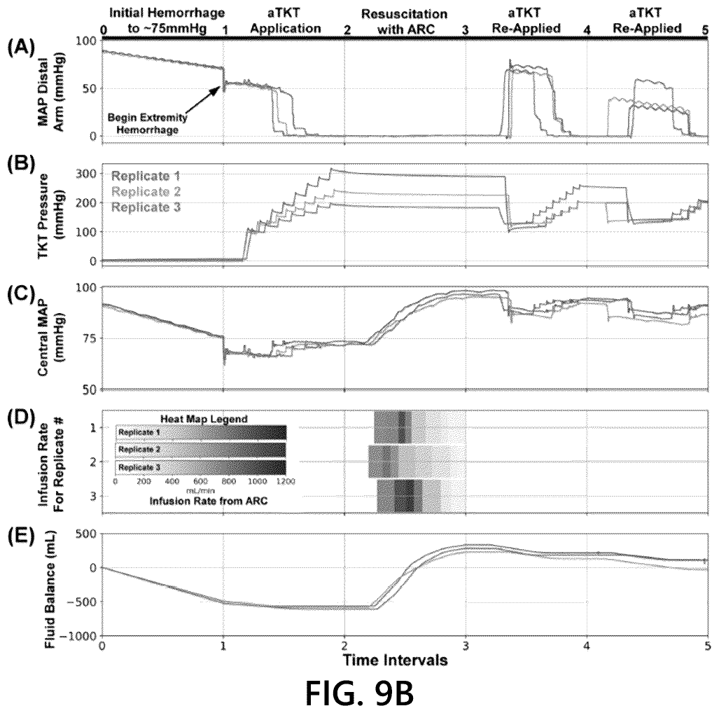

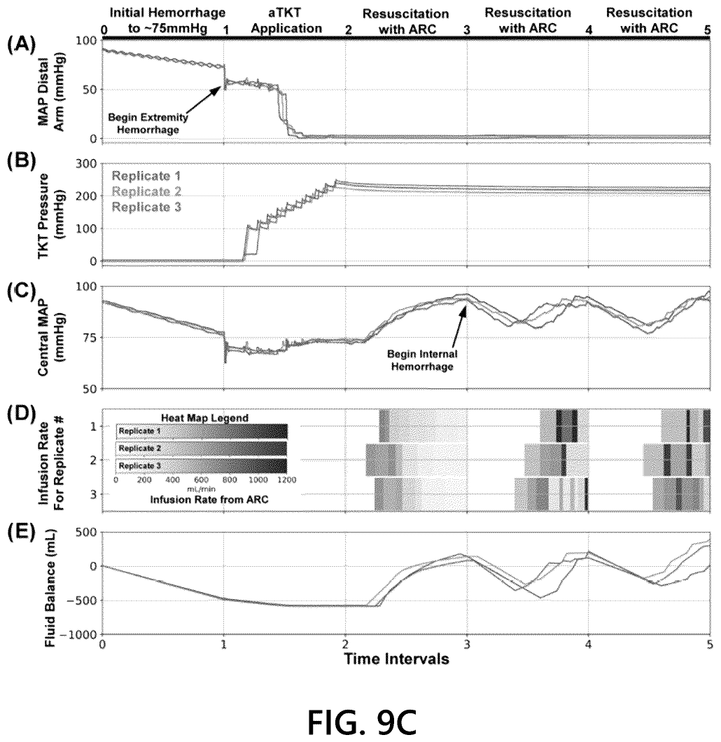

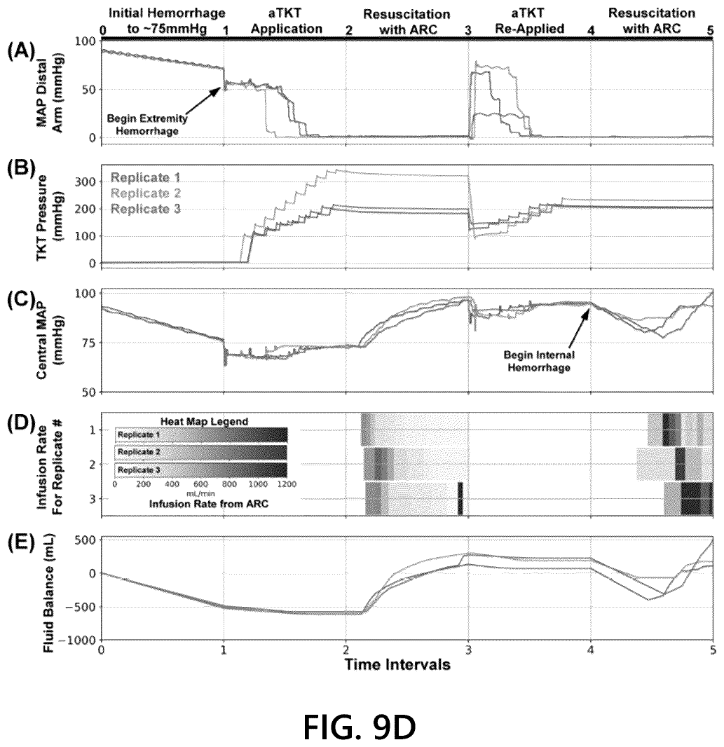

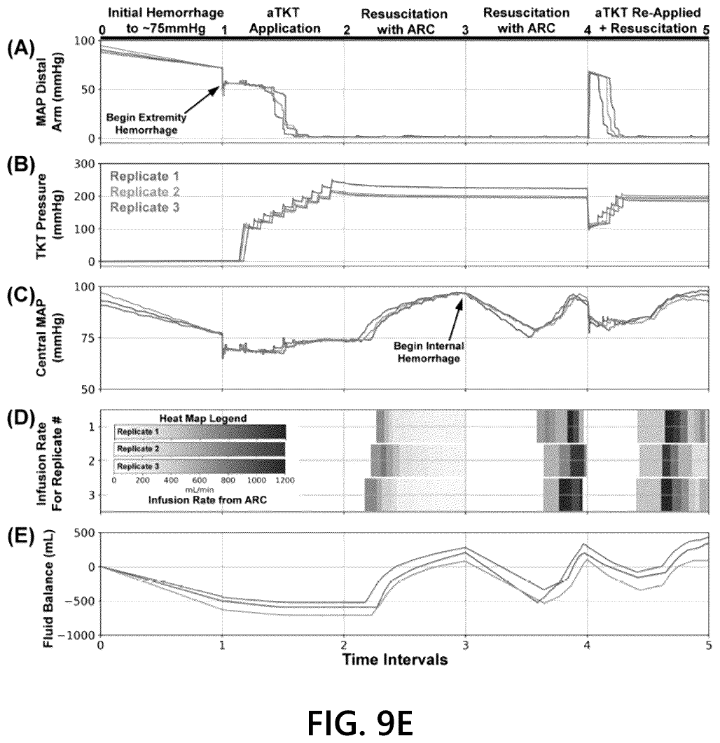

Discussion will now turn to various testing scenarios, illustrating, for different replicates, measurements such as distal mean arterial pressure, tourniquet pressure, central mean arterial pressure, infusion rate, and fluid balance. FIG. 9A depicts charts showing distal mean arterial pressure, tourniquet pressure, central mean arterial pressure, infusion rate, and fluid balance over time for a first testing scenario, wherein an initial hemorrhage (stage 1) was followed by extremity bleed and application of an automated tourniquet (stage 2) and ARC resuscitation (stage 3). FIG. 9B depicts charts showing distal mean arterial pressure, tourniquet pressure, central mean arterial pressure, infusion rate, and fluid balance over time for a second testing scenario, wherein an initial hemorrhage (stage 1) was followed by extremity bleed and application of an automated tourniquet (stage 2), ARC resuscitation (stage 3), and two stages of internal hemorrhage with ARC resuscitation (stages 4-5). FIG. 9C depicts charts showing distal mean arterial pressure, tourniquet pressure, central mean arterial pressure, infusion rate, and fluid balance over time for a third testing scenario, wherein an initial hemorrhage (stage 1) was followed by extremity bleed and application of an automated tourniquet (stage 2), ARC resuscitation (stage 3), the loosening and re-tightening of a tourniquet (stage 4), and (again) the loosening and re-tightening of a tourniquet (stage 5). FIG. 9D depicts charts showing distal mean arterial pressure, tourniquet pressure, central mean arterial pressure, infusion rate, and fluid balance over time for a fourth testing scenario, wherein an initial hemorrhage (stage 1) was followed by extremity bleed and application of an automated tourniquet (stage 2), ARC resuscitation (stage 3), the loosening and re-tightening of a tourniquet (stage 4), and then internal hemorrhage and ARC resuscitation (stage 5). FIG. 9E depicts charts showing distal mean arterial pressure, tourniquet pressure, central mean arterial pressure, infusion rate, and fluid balance over time for a fifth testing scenario, wherein an initial hemorrhage (stage 1) was followed by extremity bleed and application of an automated tourniquet (stage 2), ARC resuscitation (stage 3), internal hemorrhage and ARC resuscitation (stage 4), and then the loosening and re-tightening of a tourniquet (stage 5).

These latter charts show, with particularity, how aspects described herein are uniquely positioned to handle long-term patient management. For examples, the scenarios depicting the loosening and re-tightening of a tourniquet underscore the ability of the SACM device 101 to respond to changes in patient care and respond to those changes, even when other devices (e.g., the ARC device 102) are working.

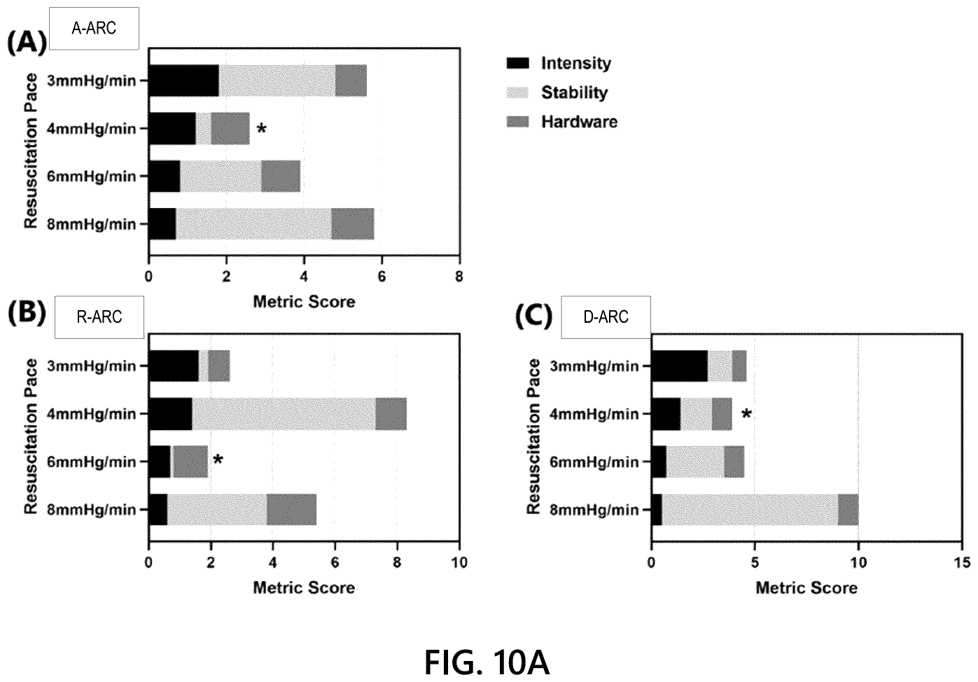

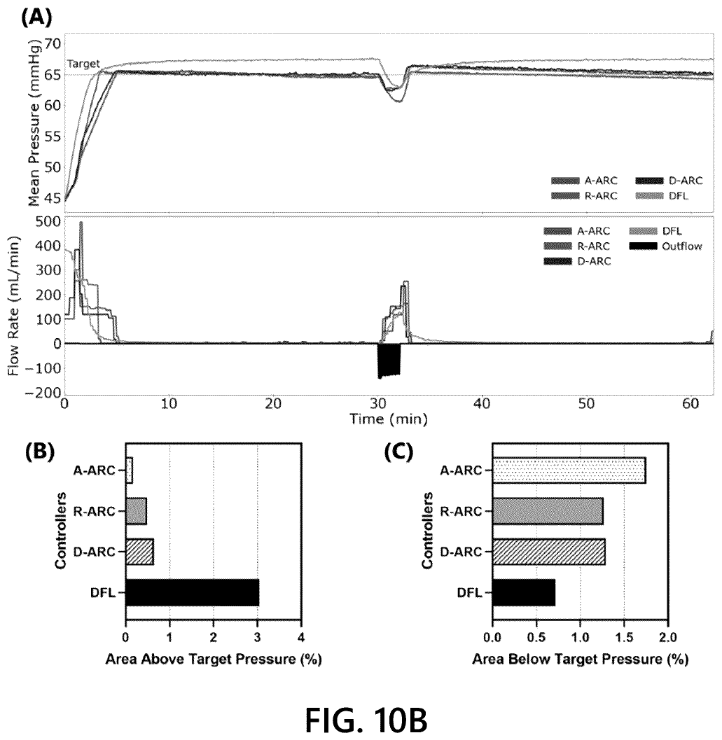

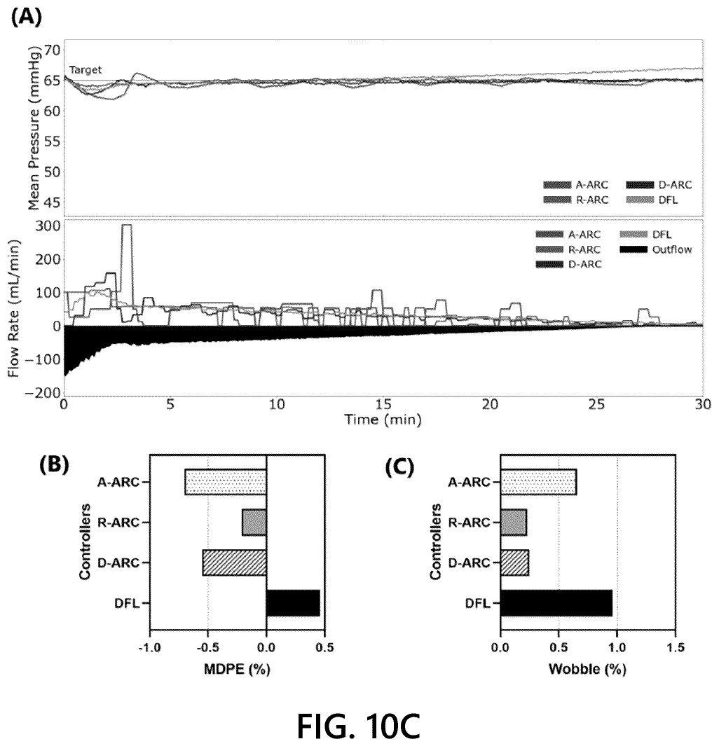

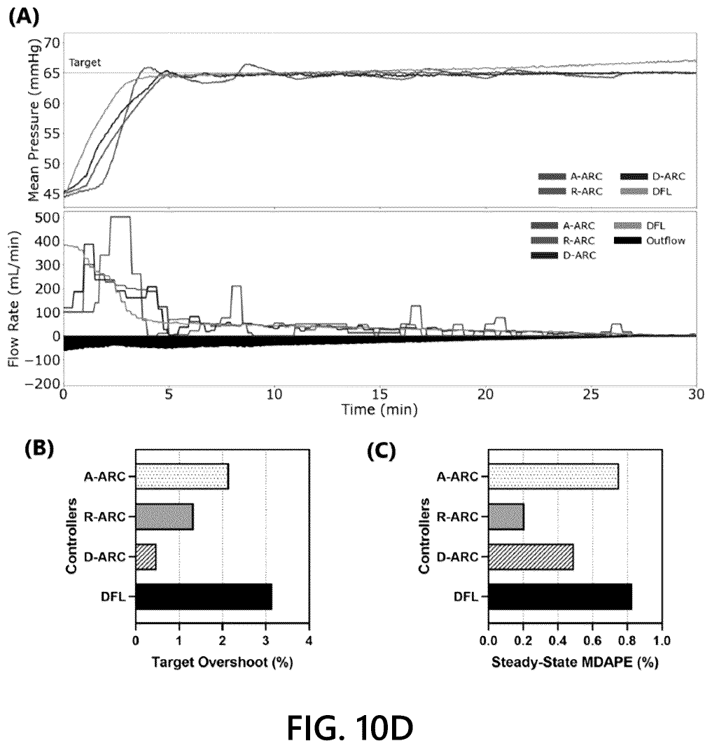

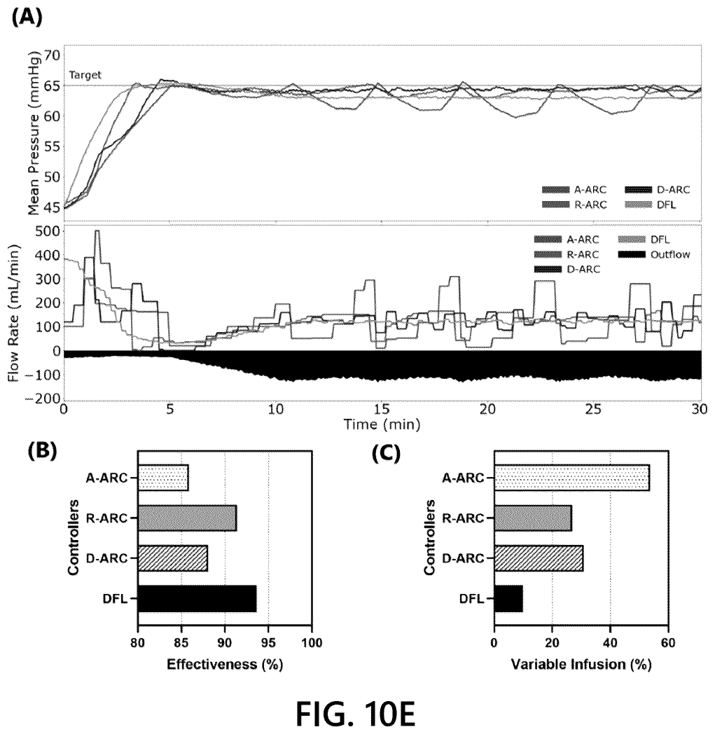

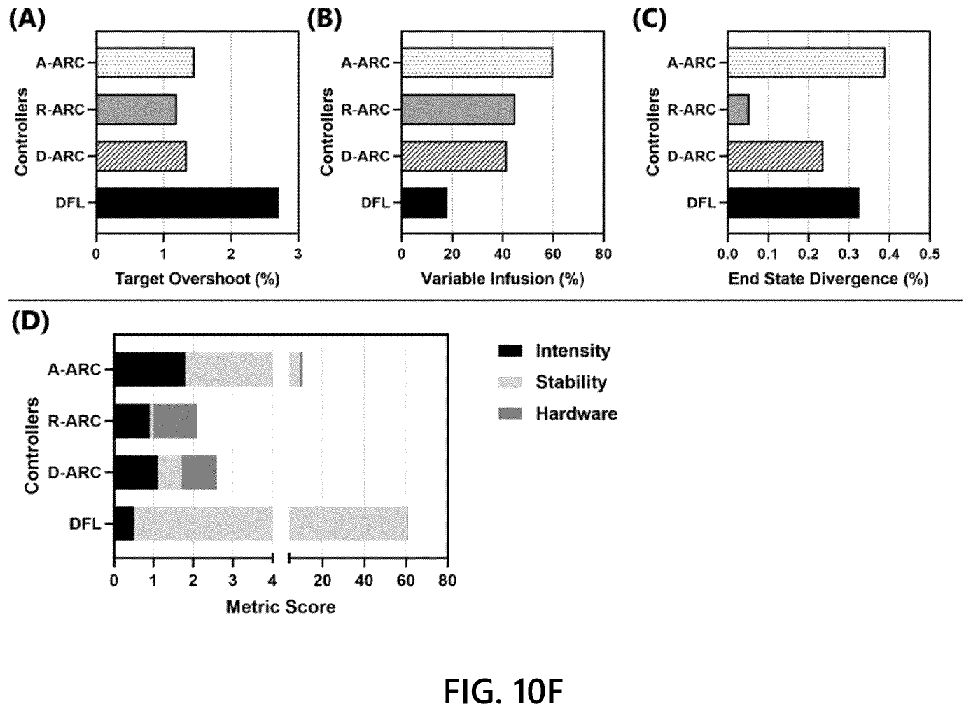

Discussion will now turn to deeper analysis of the various algorithms that may be implemented, by the SACM device 101 and/or the ARC device 102. These include, as described above, Absolute-ARC (A-ARC), which relates total fluid volume in the system to the pressure (p=m*V+P₀), Relative-ARC (R-ARC), which relates a change in volume in the system to a change in pressure (ΔP=m*ΔV+0), Dubick-o-Matic ARC (D-ARC), which relies on the Dubick constant (that is, approximately 0.337 ml/mmHg/kg), and a Dual Fuzzy Logic (DFL) approach. FIG. 10A comprises charts showing various comparisons of resuscitation paces for the A-ARC algorithm, the R-ARC algorithm, and the D-ARC algorithm, with lower values being better (e.g., faster). This chart shows that, for A-ARC, the 4 mmHg/min version performed best, for R-ARC, the 6 mmHg/min version had the lowest aggregate scores, and for D-ARC, performance was more consistent; however, the 4 mmHg/min had the lowest aggregate metric total.

Each of these algorithms were then tested using four different scenarios. In the first scenario, mean arterial pressure begins at 45 mmHg with no active bleed and a rapid hemorrhage occurs at thirty minutes and lasts for two minutes, with no additional bleed. In the second scenario, mean arterial pressure begins at 65 mmHg with an aggressive hemorrhage rate (maximum ˜125 mL/min) that slows throughout the scenario. The third scenario is similar to the second scenario, except that mean arterial pressure begins at 45 mmHg. In the fourth scenario, the mean arterial pressure begins at 45 mmHg but, after five minutes, bleeding accelerates to a maximum rate of ˜125 mL/min and holds at that rate for the remainder of the scenario. FIG. 10B shows charts indicating mean pressure and flow rate over time, as well as comparisons of various controllers with respect to target pressure, for a first scenario (mean arterial pressure begins at 45 mmHg with no active bleed and a rapid hemorrhage occurs at 30 minutes and lasts for 2 minutes, with no additional bleed). FIG. 10C shows charts indicating mean pressure and flow rate over time, as well as comparisons of various controllers with respect to target pressure, for a second scenario (mean arterial pressure begins at 65 mmHg with an aggressive hemorrhage rate (maximum ˜125 mL/min) that slows throughout the scenario). FIG. 10D shows charts indicating mean pressure and flow rate over time, as well as comparisons of various controllers with respect to target pressure, for a third scenario (similar to the second scenario, except that mean arterial pressure begins at 45 mmHg). FIG. 10E shows charts indicating mean pressure and flow rate over time, as well as comparisons of various controllers with respect to target pressure, for a fourth scenario (mean arterial pressure begins at 45 mmHg but, after five minutes, bleeding accelerates to a maximum rate of ˜125 mL/min and holds at that rate for the remainder of the scenario).

FIG. 10F comprises charts comparing various controllers in terms of overshoot, variable infusion, end-state divergence, and overall score. In this case, lower is better. As can be seen from the charts, the R-ARC algorithm generally performed in the most desirable way, having the lowest target overshoot percentage and a significantly lower end state divergence, although it did exhibit a significant degree of variability in infusion.

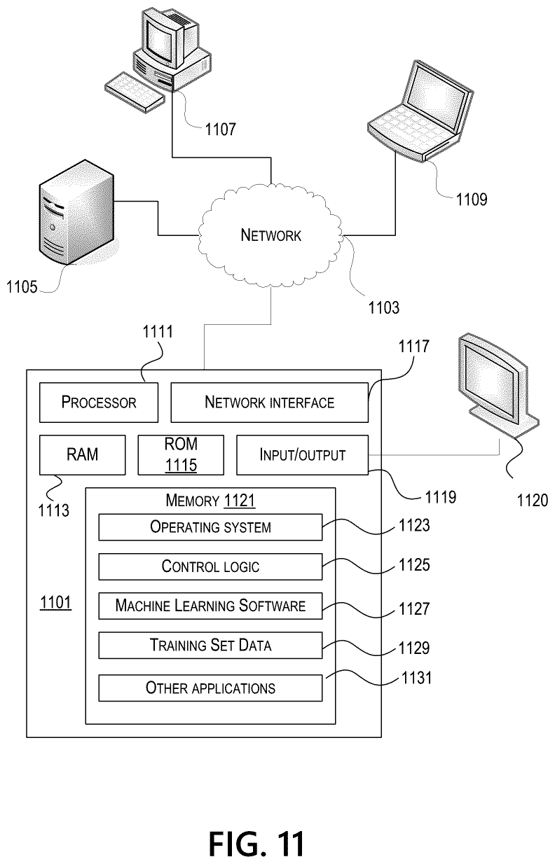

FIG. 11 illustrates one example of a computing device 1101 that may be used to implement one or more illustrative aspects discussed herein. For example, computing device 1101 may, in some embodiments, implement one or more aspects of the disclosure by reading and/or executing instructions and performing one or more actions based on the instructions. In some embodiments, computing device 1101 may represent, be incorporated in, and/or include various devices such as a desktop computer, a computer server, a mobile device (e.g., a laptop computer, a tablet computer, a smart phone, any other types of mobile computing devices, and the like), and/or any other type of data processing device.

Computing device 1101 may, in some embodiments, operate in a standalone environment. In others, computing device 1101 may operate in a networked environment. As shown in FIG. 11, computing devices 1101, 1105, 1107, and 1109 may be interconnected via a network 1103, such as the Internet. Other networks may also or alternatively be used, including private intranets, corporate networks, LANs, wireless networks, personal networks (PAN), and the like. Network 1103 is for illustration purposes and may be replaced with fewer or additional computer networks. A local area network (LAN) may have one or more of any known LAN topologies and may use one or more of a variety of different protocols, such as Ethernet. Devices 1101, 1105, 1107, 1109 and other devices (not shown) may be connected to one or more of the networks via twisted pair wires, coaxial cable, fiber optics, radio waves or other communication media.

As seen in FIG. 11, computing device 1101 may include a processor 1111, RAM 1113, ROM 1115, network interface 1117, input/output interfaces 1119 (e.g., keyboard, mouse, display, printer, etc.), and memory 1121. Processor 1111 may include one or more computer processing units (CPUs), graphical processing units (GPUs), and/or other processing units such as a processor adapted to perform computations associated with machine learning. I/O 1119 may include a variety of interface units and drives for reading, writing, displaying, and/or printing data or files. I/O 1119 may be coupled with a display such as display 1120. Memory 1121 may store software for configuring computing device 1101 into a special purpose computing device in order to perform one or more of the various functions discussed herein. Memory 1121 may store operating system software 1123 for controlling overall operation of computing device 1101, control logic 1125 for instructing computing device 1101 to perform aspects discussed herein, machine learning software 1127, training set data 1129, and other applications 1131. Control logic 1125 may be incorporated in and may be a part of machine learning software 1127. In other embodiments, computing device 1101 may include two or more of any and/or all of these components (e.g., two or more processors, two or more memories, etc.) and/or other components and/or subsystems not illustrated here.

Devices 1105, 1107, 1109 may have similar or different architecture as described with respect to computing device 1101. Those of skill in the art will appreciate that the functionality of computing device 1101 (or device 1105, 1107, 1109) as described herein may be spread across multiple data processing devices, for example, to distribute processing load across multiple computers, to segregate transactions based on geographic location, user access level, quality of service (QOS), etc. For example, computing devices 1101, 1105, 1107, 1109, and others may operate in concert to provide parallel computing features in support of the operation of control logic 1125 and/or machine learning software 1127.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a data processing system, or a computer program product.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method configured to provide adaptive and closed loop hemorrhagic shock resuscitation using an automated tourniquet and an adaptive resuscitation controller, the method comprising:
causing activation of the adaptive resuscitation controller by:
determining a desired mean arterial pressure setpoint; and
causing, based on the desired mean arterial pressure setpoint, the adaptive resuscitation controller to infuse a first quantity of fluids into a patient;
monitoring blood pressure parameters of the patient; and
based on comparing the blood pressure parameters to the desired mean arterial pressure setpoint, causing the adaptive resuscitation controller to infuse a second quantity of fluids into the patient.

2. The method of claim 1, wherein the determining the desired mean arterial pressure setpoint comprises:
determining, based on the desired mean arterial pressure setpoint, a plurality of different intermediate mean arterial pressure setpoints,
wherein the causing the adaptive resuscitation controller to infuse the first quantity of fluids into the patient is based on a first intermediate mean arterial pressure setpoint of the plurality of different intermediate mean arterial pressure setpoints, and
wherein the causing the adaptive resuscitation controller to infuse the second quantity of fluids into the patient is based on a second intermediate mean arterial pressure setpoint of the plurality of different intermediate mean arterial pressure setpoints.

3. The method of claim 1, further comprising:
determining a correction factor based on a difference between:
a predicted time of an increase in mean arterial pressure, and
an actual time of an increase in mean arterial pressure indicated by the blood pressure parameters of the patient,
wherein the causing the adaptive resuscitation controller to infuse the second quantity of fluids into the patient is based on the correction factor.

4. The method of claim 1, wherein the causing the adaptive resuscitation controller to infuse the second quantity of fluids into the patient comprises:
determining a volume-pressure relationship between:
one or more of:
a total fluid volume associated with the patient; or
a volume of the first quantity of fluids; and
at least one of the blood pressure parameters, and
determining, based on the volume-pressure relationship, a volume of the second quantity of fluids.

5. The method of claim 1, wherein the comparing the blood pressure parameters to the desired mean arterial pressure setpoint comprises:
determining, based on a Dubick constant of approximately 0.337 ml/mmHg/kg, a volume of the second quantity of fluids.

6. The method of claim 1, further comprising:
causing activation of the automated tourniquet by causing the automated tourniquet to increase pressure of a component of the automated tourniquet to impede extremity bleeding of a patient;
monitoring component pressure measurements corresponding to the component of the automated tourniquet; and
based on the component pressure measurements, causing modification of one or more operating parameters of the automated tourniquet by:
detecting air pressure oscillations in the component; and
increasing a volume of air in the component.

7. The method of claim 6, wherein the causing the adaptive resuscitation controller to infuse the second quantity of fluids into the patient comprises causing the adaptive resuscitation controller to infuse the second quantity of fluids into the patient at the same time as causing modification of the one or more operating parameters of the automated tourniquet.

8. The method of claim 6, further comprising:
detecting, based on the component pressure measurements, hemorrhage associated with a first appendage of the patient, wherein the automated tourniquet is configured to impede extremity bleeding of a second appendage of the patient; and
causing the adaptive resuscitation controller to cease infusion of fluids.

9. The method of claim 6, wherein the causing the adaptive resuscitation controller to infuse the second quantity of fluids into the patient is further based on the component pressure measurements.

10. The method of claim 6, wherein the causing modification of the one or more operating parameters of the automated tourniquet is further based on the blood pressure parameters of the patient.

11. The method of claim 1, wherein the causing activation of the automated tourniquet comprises deactivating an automatic functioning of the automated tourniquet, and wherein causing activation of the adaptive resuscitation controller comprises deactivating an automatic functioning of the adaptive resuscitation controller.

12. The method of claim 1, further comprising:
based on determining that the blood pressure parameters are within a user-defined error level of the desired mean arterial pressure setpoint, causing the adaptive resuscitation controller to cease infusion of fluids.

13. A computing device configured to provide adaptive and closed loop hemorrhagic shock resuscitation using an adaptive resuscitation controller, the computing device comprising:
one or more processors; and
memory storing instructions that, when executed by the one or more processors, cause the computing device to:
cause activation of the adaptive resuscitation controller by:
determining a desired mean arterial pressure setpoint; and
causing, based on the desired mean arterial pressure setpoint, the adaptive resuscitation controller to infuse a first quantity of fluids into a patient;
monitor blood pressure parameters of the patient; and
based on comparing the blood pressure parameters to the desired mean arterial pressure setpoint, cause the adaptive resuscitation controller to infuse a second quantity of fluids into the patient.

14. The computing device of claim 13, wherein the instructions, when executed by the one or more processors, cause the computing device to determine the desired mean arterial pressure setpoint by causing the computing device to:
determine, based on the desired mean arterial pressure setpoint, a plurality of different intermediate mean arterial pressure setpoints,
wherein the instructions, when executed by the one or more processors, cause the computing device to cause the adaptive resuscitation controller to infuse the first quantity of fluids into the patient based on a first intermediate mean arterial pressure setpoint of the plurality of different intermediate mean arterial pressure setpoints, and
wherein the instructions, when executed by the one or more processors, cause the computing device to cause the adaptive resuscitation controller to infuse the second quantity of fluids into the patient based on a second intermediate mean arterial pressure setpoint of the plurality of different intermediate mean arterial pressure setpoints.

15. The computing device of claim 13, wherein the instructions, when executed by the one or more processors, cause the computing device to:
determine a correction factor based on a difference between:
a predicted time of an increase in mean arterial pressure, and
an actual time of an increase in mean arterial pressure indicated by the blood pressure parameters of the patient,
wherein the instructions, when executed by the one or more processors, cause the computing device to cause the adaptive resuscitation controller to infuse the second quantity of fluids into the patient based on the correction factor.

16. The computing device of claim 13, wherein the instructions, when executed by the one or more processors, cause the computing device to cause the adaptive resuscitation controller to infuse the second quantity of fluids into the patient by causing the computing device to:
determine a volume-pressure relationship between:
one or more of:
a total fluid volume associated with the patient; or
a volume of the first quantity of fluids; and
at least one of the blood pressure parameters, and
determine, based on the volume-pressure relationship, a volume of the second quantity of fluids.

17. A system comprising:
a computing device for providing adaptive and closed loop hemorrhagic shock resuscitation, and
an adaptive resuscitation controller,
wherein the computing device is configured to:
cause activation of the adaptive resuscitation controller by:
determining a desired mean arterial pressure setpoint; and
causing, based on the desired mean arterial pressure setpoint, the adaptive resuscitation controller to infuse a first quantity of fluids into a patient;
monitor blood pressure parameters of the patient; and
based on comparing the blood pressure parameters to the desired mean arterial pressure setpoint, cause the adaptive resuscitation controller to infuse a second quantity of fluids into the patient.

18. The system of claim 17, wherein the computing device is configured to determine the desired mean arterial pressure setpoint by:
determining, based on the desired mean arterial pressure setpoint, a plurality of different intermediate mean arterial pressure setpoints,
wherein the computing device is configured to cause the adaptive resuscitation controller to infuse the first quantity of fluids into the patient based on a first intermediate mean arterial pressure setpoint of the plurality of different intermediate mean arterial pressure setpoints, and
wherein the computing device is configured to cause the adaptive resuscitation controller to infuse the second quantity of fluids into the patient based on a second intermediate mean arterial pressure setpoint of the plurality of different intermediate mean arterial pressure setpoints.

19. The system of claim 17, wherein the computing device is further configured to:
determine a correction factor based on a difference between:
a predicted time of an increase in mean arterial pressure, and
an actual time of an increase in mean arterial pressure indicated by the blood pressure parameters of the patient,
wherein the computing device is configured to cause the adaptive resuscitation controller to infuse the second quantity of fluids into the patient based on the correction factor.

20. The system of claim 17, wherein the computing device is configured to cause the adaptive resuscitation controller to infuse the second quantity of fluids into the patient by:
determining a volume-pressure relationship between:
one or more of:
a total fluid volume associated with the patient; or
a volume of the first quantity of fluids; and
at least one of the blood pressure parameters, and
determining, based on the volume-pressure relationship, a volume of the second quantity of fluids.
